(19) United States
(12) Patent Application Publication
(10) Pub. No.: US 2011/0040290 A1
(43) Pub. Date: Feb. 17, 2011

(54) Threaded urinary catheter

(76) Inventors: Filiberto Zadini, Northridge, CA (US); Giorgio Zadini, Santa Rosa Valley, CA (US)

Correspondence Address: Filiberto Zadini, 16874 Rayen, Northridge, CA, 91343, US

(21) Appl. No.: 12/806,584

(22) Filed: Aug. 17, 2010

Related U.S. Application Data
(60) Provisional application No. 61/274,423, filed on Aug. 17, 2009.

(51) Int. Cl. A61M 27/00

(52) U.S. Cl. 604/544

(57) ABSTRACT

A novel urinary catheter having an helicoidally threaded , screw shaped tip to facilitate insertion of the catheter tip by rotation into the bladder, especially useful in situation of difficult passage and entry into the bladder through a stenotic prostatic urethra. The operator instead of advancing the catheter by axial loading as it is presently done with ordinary catheters will rotate the urinary catheter along its longitudinal axis . Rotation of the catheter is transmitted to its specially designed screw shaped tip facilitating its engagement, advancement and insertion into the bladder through a stenotic segment of the urethral canal.

### RELATED MATTER

This application claims priority to U.S. Provisional Patent Application No. 61/274,423 filed on Aug. 17, 2009, and entitled “Threaded Urinary Catheter”, the relevant content of which is hereby incorporated by reference.

### FIELD OF INVENTION

This application relates to urinary catheters more precisely to urinary catheters provided with ways and means which facilitate their insertion into the bladder through the urethra.

### BACKGROUND OF THE INVENTION

A urinary catheter is a flexible tube usually made of natural rubber or silicone, such a tube having a tip which is inserted into the urethra via the urethral meatus. The presently used ordinary urinary catheters are advanced through the urethra into a patient's bladder solely via axial loading. There are few different types of urinary catheters:

A Foley catheter, the most common, is provided at its tip with an inflatable balloon which secures the catheter in position within the bladder not allowing its expulsion from the bladder. The balloon at the tip is inflated with sterile water or normal saline. Foleys are commonly made of silicone or rubber.

A Robinson catheter is a flexible catheter used for short term drainage of urine. Unlike the Foley catheter, it has no balloon on its tip and therefore cannot stay in place. A Coude catheter is generally more rigid than a Foley catheter . It has a curved tip, the purpose of which is to facilitate its insertion thru urethral canal strictures such as in the case of benign prostatic hypertrophy.

A Coudé catheter may be provided with a balloon or not. A three ways irrigation catheter has a separate lumen to carry irrigation fluid into the bladder. It is mostly used to irrigate the bladder in case of hematuria with or without presence of clots within the bladder.

The most common urinary catheter diameter sizes are 10 F (3.3 mm) to 28 F (9.3 mm).The catheter tube is usually made of rubber or silicone. The length of urinary catheters varies. It is approximately 40 cm.

Common indications for placing urinary catheter in a patient include:
1) acute or chronic urinary retention, both mechanical such as in the case of benign prostatic hypertrophy or not mechanical such as in spastic bladder neck,
2) the need to measure the urine output in critical care patients
3) incontinence,
4) patients status post bladder surgery

In some cases the urinary catheter cannot be advanced in the urethra due to anatomical or functional restrictions. Presently if a urinary catheter cannot be advanced into the bladder by axial loading due to anatomical and functional restrictions, the only options left to the treating physician are the use of urethral dilators which being rigid can cause significant trauma to the urethral canal or to perform supra-pubic urine needle aspiration which can carry significant risks/complications including bowel puncturing, infection and/or bleeding.

The design and structure of the urinary catheter have basically not changed since inception. Applicants have searched the Patent Office and they have not found any patent or patent application which address the difficulties of catheter insertion into the bladder due urethral and/or bladder anatomical and/or functional restrictions in a simple and straightforward way such as the way Applicants are disclosing in this Application maintaining the basic urinary catheter design and structure unchanged except for the tip design. Specifically Applicants have found no references to urinary catheters with screw shaped tip advanced by rotation within the narrowed urethral segment.

### BRIEF SUMMARY OF THE INVENTION

With the present Provisional Patent Application, Applicants disclose a novel type of urinary catheter, a urinary catheter provided with a threaded tip, screw shaped, which facilitates insertion of the urinary catheter into the bladder by rotation and not by axial loading ,whenever the urethra is stenotic and catheter advancement is problematic, traumatic and/or unsuccessful. Applicants catheter is designed to maximize patient comfort and to reduce urethral trauma. As above indicated Applicants catheter is advanced by rotation and not by axial loading as in the case of all the present catheters.

### OBJECTS OF THE INVENTION

It is an object of the present invention to provide a novel urinary catheter which by design and structure is easier to insert into the bladder than the presently used catheters. Applicants catheter is inserted into the bladder by rotational advancement and not by axial loading overcoming in a simpler and more effective manner the stenotic urethral segment.

It is an object of the present invention to provide a urinary catheter that by design and structure is less traumatic to insert providing better comfort and less trauma than the present catheters.

It is an object of the present invention to provide the health operator with a urinary catheter that allows easier and less traumatic successful insertion of its tip into the bladder making the use of urethral dilators and the procedure of supra-pubic needle aspiration which both carry significant risks, obsolete.

### FIGURES

FIG. 1 is a an front view of Applicants urinary catheter formed with an helicoidally shaped tip.

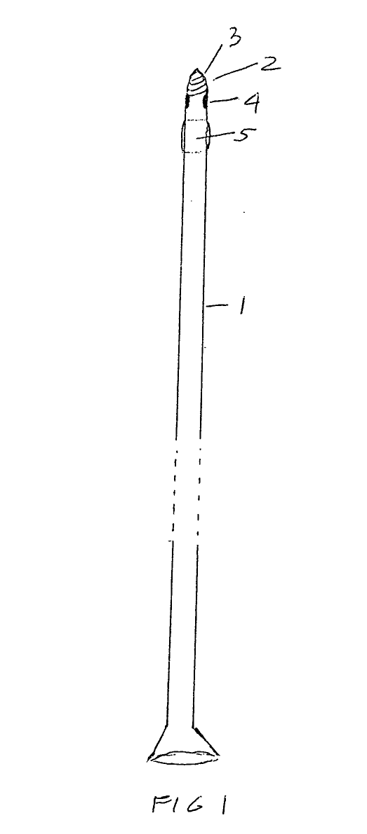

FIG. 2 is an exploded front view of the threaded catheter tip, screw shaped.

FIG. 3 is an exploded front view showing another embodiment of the urinary catheter tip of FIG. 1 in which the thread is proximal to the catheter tip orifices.

FIG. 4 is an exploded front view showing another embodiment of the urinary catheter tip of in which the catheter tip is grossly conical to facilitate engagement of the catheter tip with a restricted urethral canal.

FIG. 5 is a schematic cross section view of an hypertrophic prostate gland with Applicants screw shaped tip of the urinary catheter in place, being advanced by rotation.

### SPECIFICATIONS

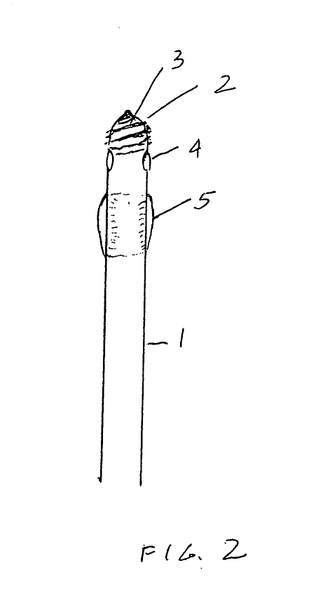

The preferred embodiment of this invention consists of urinary catheter 1 provided with an helicoidally threaded tip or screw shaped tip 2. As better shown in FIG. 2, urinary catheter tip 2 is formed with helicoidally shaped flanges or thread or screw means 3, at its distal end, distally to orifices 4. In FIG. 2 tip 2 is also formed with balloon 5 but urinary catheter 1 can be also be void of balloon 5.

More than one thread can be placed at different level of catheter 1, being the diameter of each thread just larger of a fraction than the diameter of catheter 1.

Tip 2 of urinary catheter 1 due to its intrinsic flexibility secondary to the material used, i.e. rubber or silicone or the likes can be not only straight but also curved as in the Coude' type of catherers. Any presently used urinary catheter with balloon or not at their distal end, can be formed with threaded tip 2: Foley, Coude', Robertson and three ways irrigation catheter. In use, urinary catheter tip 2 of urinary catheter 1 is lubricated preferably with lidocaine 2% jelly. Catheter tip 2 is inserted into the urethral meatus of a patient and catheter 1 is advanced through the urethral canal to enter the bladder with tip 2. In case of narrowing of the urethral canal at any level of the urethra, either anatomical or functional, catheter 1 is rotated in clockwise manner. Catheter 1 can be made sufficiently rigid to sustain the torque mechanism.

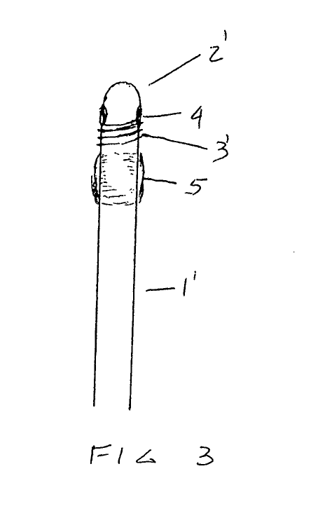

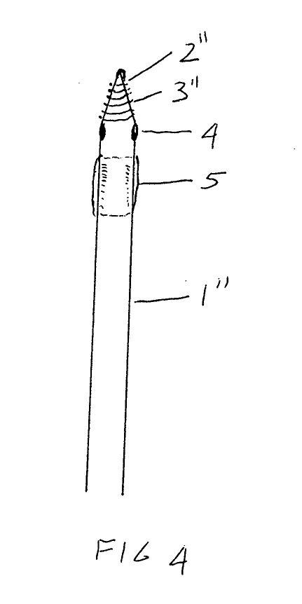

The rotation of catheter 1 along its longitudinal axis will screw tip 2 into the bladder along the urethra overcoming the restriction/obstruction within the urethral canal, making its own passageway through the narrowed segment of the urethra. If catheter 1 is not sufficiently rigid to sustain the torque mechanism, the rotation of catheter 1 will result in a twisting of a segment of catheter proximal to the threaded segment. The twisting of the catheter segment proximal to the threaded segment will result in charge of torque energy upon the threaded segment to a point that the stored torque energy is released after few twists causing rotation of threaded tip 2 within the urethra facilitating advancement and introduction of catheter tip 2 into the bladder of a patient. FIG. 3 shows another embodiment of urinary catheter 1, urinary catheter 1′, where tip 2′ is formed with thread 3′ proximal to orifices 4. Thread 3′ can also be located just proximally to balloon 5. As shown in FIG. 4, tip 2″ of catheter 1″can be shaped as a cone to facilitate its engagement with narrowed urethral segments.

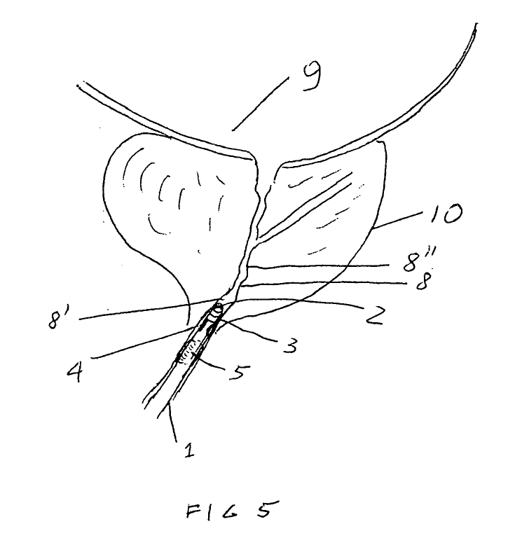

FIG. 5 is a schematic exploded cross section view of the of the prostatic urethra of a patient suffering from Benign Prostatic Hypertrophy with screw shaped tip 2 of urinary catheter 1 engaged in the prostatic urethra. Prostate gland 10 is significantly enlarged to the point that prostatic urethra 8 is compressed and distorted just below bladder 9 reducing lumen 8′ of prostatic urethra 8″ of urethra 7 . Such a narrowing or restriction or narrowed or restricted segment 8″ of prostatic urethra 8, if moderate or severe, does not allow the passage by axial loading of ordinary urinary catheters into bladder 9. Instead applicants urinary catheter 1, provided with threaded tip 3, when rotated clockwise will advance by rotation more easily through restricted urethral segment 8″ than if advanced by axial loading. Screw shaped tip 2 will advance by means of rotation within narrowed segment 8″ of prostatic urethra 8 making its own passageway thru the narrowed urethral segment 8″. Once catheter tip 2 is position within bladder 9, bladder 9 can be evacuated of its content i.e. urine or can be irrigated with normal saline or water to evacuate blood and/or clots.

What we claim is:

1. A urinary catheter insertable into a patient's urethral canal, comprising:
a distal tip segment and
a tubular shaft segment extending from said tip segment to a proximal end, said distal segment being provided on its outer surface with helicoidally shaped flanges to enable rotational advancement of the distal tip segment along the urethral canal via torque rotation applied by an operator upon said tubular shaft, said tubular shaft being sufficiently rigid to sustain torque and transfer it to said distal tip segment.

2. A urinary catheter comprising:
a flexible tube and
a screw shaped distal tip to permit advancement of said catheter by rotation into a bladder of a patient through a narrowed urethral prostatic segment.

3. The urinary catheter of claim 2, wherein the screw shaped tip is formed with more than one helicoidal flange of increasing diameter from a first anterior flange.

4. A method of inserting a urinary catheter provided with a screw shaped tip into a urethra of a patient comprising unidirectional rotation of the urinary catheter being applied by an operator, to confer to said urinary catheter a torque sufficient to be transmitted to the screw shaped tip to allow advancement by unidirectional rotation into a narrowed prostatic segment.

5. The urinary catheter of claim 2 formed with at least two orifices for drainage of urine from a bladder of a patient and a balloon for securing catheter tip placement in the bladder and for avoiding removal wherein the screw shaped tip is posterior to the balloon.

6. The urinary catheter of claim 2 formed with at least two orifices for drainage of urine from a bladder of a patient and a balloon for securing catheter tip placement in the bladder and for avoiding removal wherein the screw shaped tip is anterior to the balloon
